(19) United States
(12) Patent Application Publication
(10) Pub. No.: US 2026/0115041 A1
(43) Pub. Date: Apr. 30, 2026

(54) SURGICAL HANDPIECE WITH CONSTRICTIONS IN ASPIRATION PATHWAY

(71) Applicant: Johnson & Johnson Surgical Vision, Inc., Irvine, CA (US)

(72) Inventor: Mark Delsman, Irvine, CA (US)

(21) Appl. No.: 18/926,290

(22) Filed: Oct. 24, 2024

(51) Int. Cl. A61F 9/007; A61M 1/00

(52) U.S. Cl. CPC A61F 9/00745; A61M 1/76

(57) ABSTRACT

A surgical handpiece is provided herein. The surgical handpiece may include: an ultrasonic horn having a lumen and configured to couple with the needle; one or more piezoelectric elements coupled with the horn and configured to ultrasonically vibrate a distal end of the needle to emulsify material; an aspiration channel extending from the distal end of the needle and through the lumen of the horn creating an aspiration pathway; and at least one constriction on an inner surface of the lumen of the horn and into the aspiration pathway, wherein the at least one constriction is configured to emulsify the material proximally along the aspiration pathway from the distal end of the needle in response to the one or more piezoelectric elements.

### FIELD OF THE DISCLOSURE

This disclosure relates generally to surgical handpieces, and more particularly to surgical handpieces having constrictions in the aspiration pathway.

### BACKGROUND

A cataract is a clouding and hardening of the eye's natural lens. Phacoemulsification cataract surgery is a common procedure to treat a cataract. In this procedure, a clear cornea or scleral incision is made in the eye to access its internal structures, and a portion of the anterior surface of the lens capsule is removed to gain access to the cataract. The surgeon then inserts a needle of an ultrasonic handpiece into the cataract. The tip of the needle vibrates at an ultrasonic frequency to emulsify the cataract lens. Simultaneously, a pump aspirates emulsified particles and fluid from the eye through the tip of the needle, and the aspirated fluids are replaced with an irrigation of a balanced salt solution via a sleeve that at least partially surrounds the needle of the handpiece to maintain intraocular pressure in the anterior chamber of the eye.

However, lens particles aspirated during cataract surgery can cause clogging at various locations within the fluidics system, such as at fittings, tubing connections, or other areas if the fluid path becomes constricted. Such zones of constriction may occur all along the fluidics path, from the phacoemulsification tip to the phacoemulsification console. In some cases, such clogs are caused or exacerbated by cylindrical cores that are aspirated through the needle tip, but which then tumble and lodge sideways within the fluidics path.

To address these clogs, fluid channels are typically designed with larger diameters, and transitions between components are designed to minimize the risk of clogging. However, larger fluid channels can have a detrimental impact on other aspects of fluidic performance, such as pressurization and responsiveness upon vacuum adjustment.

### BRIEF DESCRIPTION OF THE DRAWINGS

The following drawings are illustrative of particular examples of the present disclosure and therefore do not limit the scope of the present disclosure. The drawings are not to scale and are intended for use in conjunction with the explanations in the following detailed description. In the drawings, like reference numerals may represent like parts and assemblies throughout the several views.

FIG. 1 illustrates an exemplary handpiece of a surgical system;

FIG. 2 illustrates an exemplary handpiece of a surgical system;

FIG. 3 illustrates an exemplary central lumen of a surgical handpiece;

FIGS. 4A and 4B illustrate exemplary central lumens of a surgical handpiece; and

FIG. 5 illustrates an exemplary handpiece of a surgical system.

### DETAILED DESCRIPTION

The following discussion omits or only briefly describes conventional features of surgical handpieces that are apparent to those skilled in the art. Those of ordinary skill may thus recognize that other elements may be desirable and/or necessary to implement the devices, systems, and methods described herein. It is noted that various examples are described in detail with reference to the drawings. Reference to these various examples does not limit the scope of the claims attached hereto. Additionally, any examples set forth in this specification are intended to be non-limiting and merely set forth some of the many possible implementations for the appended claims. Further, particular features described herein can be used in combination with other described features in each of the various possible combinations and permutations. As such, it is understood that the detailed description is exemplary and explanatory only and is not restrictive of the broad inventive concepts upon which the examples disclosed herein are based.

Unless otherwise specifically defined herein, all terms are to be given their broadest reasonable interpretation. This includes meanings implied from the specification as well as meanings understood by those skilled in the art and/or as defined in dictionaries, treatises, etc.

It is noted that, as used in the specification and the appended claims, the singular forms “a,” “an” and “the” include plural referents unless otherwise specified. The terms “includes” and/or “including,” when used in this specification, specify the presence of stated features, elements, and/or components, but do not preclude the presence or addition of one or more other features, steps, operations, elements, components, and/or groups thereof.

Relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then-described or as shown in the drawing figure under discussion. These relative terms are for convenience of description and normally are not intended to require a particular orientation in actuality. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The phrases “configured to”, “operatively” or “operably connected” indicates such an attachment, coupling or connection that allows the pertinent structures to operate as intended by virtue of that relationship.

Reference throughout the specification to “exemplary”, “one example”, “an example” or “some examples” means that a particular feature, structure, or characteristic is described in connection with at least one example of the subject matter disclosed. Thus, the appearance of the phrases “in one example”, “in an example”, “by way of example”, “in some examples”, and other like-phrases in various places throughout the specification do not necessarily refer to a single or the same example. Further, the particular features, structures or characteristics of “one example”, “an example”, “some examples”, or other like-phrases may be combined in any suitable manner with each other to form additional examples of such combinations. It is intended that examples of the disclosed subject matter cover modifications and variations thereof. Terms such as “first,” “second,” “third,” etc., merely identify one of a number of portions, components, steps, operations, functions, and/or points of reference as disclosed herein, and likewise do not necessarily limit examples of the present disclosure to any particular configuration or orientation.

Moreover, throughout this disclosure, various aspects may be presented in a range format. It should be understood that a description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range. In relation to a range and as used herein, the term “about” in reference to a measurable value, such as an amount, a temporal duration, and the like, is meant to encompass variations of plus or minus 20%, plus or minus 10%, plus or minus 5%, plus or minus 1%, and plus or minus 0.1% of the specified value, as such variations are appropriate.

The terms “proximal,” “distal,” “anterior,” “posterior,” “medial,” “lateral,” “superior,” and “inferior” are defined by their standard usage indicating a directional term of reference. For example, “proximal” refers to a position that is situated nearer to the center of a body or point of attachment or interest, while “distal” refers to a position that is situated away from the center of the body or point of attachment or interest. In another example, “anterior” refers to the front of a body or structure, while “posterior” refers to the rear of a body or structure, in relation to a relative viewpoint. In another example, “medial” refers to the direction towards the midline of a body or structure, and “lateral” refers to the direction away from the midline of a body or structure. In some examples, “lateral” or “laterally” may refer to any sideways direction. In another example, “superior” refers to the top of a body or structure, while “inferior” refers to the bottom of a body or structure. It should be understood, however, that the directional term of reference may be interpreted within the context of a specific body or structure, such that a directional term referring to a location in the context of the reference body or structure may remain consistent as the orientation of the body or structure changes.

By providing constriction areas/zones in a central aspiration lumen that are affected by, or otherwise configured to receive energy, such as vibrational energy, from, the ultrasonic transducing element(s) of a surgical handpiece, aspirated particles, such as aspirated emulsified eye lens particles, may undergo a secondary emulsification without the addition of significant obstruction in the fluidic path. This secondary emulsification may further reduce the size and aspect ratio of the aspirated particles, and may thus reduce the risk of clogging downstream. Accordingly, the devices, systems and methods described herein demonstrate unexpected results of, rather than impeding flow, instead improving flow through the aspiration lumen of a surgical handpiece by reducing or constricting at least one region, or “zone”, of the aspiration lumen during the delivery of ultrasonic energy.

The high speed expansion and contraction of the aspiration pathway(s) caused by the ultrasonics may temporarily capture and hold particles within these constriction zones. This holding of the particles in the constriction zones allows repeated acting of the ultrasonic vibrations upon the particles, thus reducing their size. Moreover, the expansion and contraction forces not only hold the particles for continued action thereupon by the ultrasonics, but also provide a continuous application and release of “crushing” forces by designed constrictions as the expansion and contraction of the aspiration pathway itself acts upon the particles within the constriction zone(s). Accordingly, the disclosure provides additional emulsification of particles in any surgical context in which aspiration of emulsified particles is used.

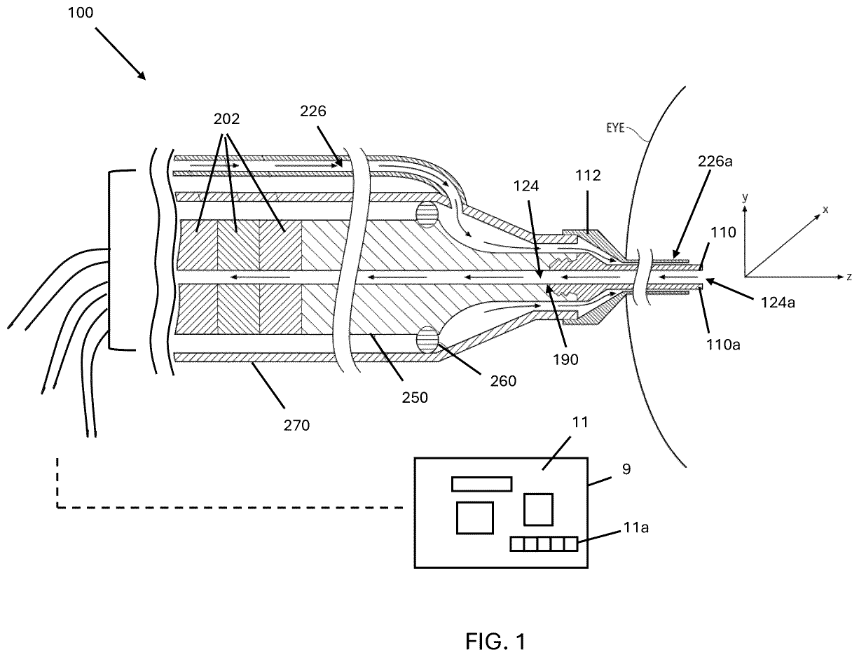

With reference now to FIG. 1, a cross-section along the longitudinal axis of a portion of a handpiece, such as a phacoemulsification handpiece 100, for use in a surgical system 1, is shown. Generally, the handpiece 100 includes a needle 110 having needle tip, or “phaco tip”, 110a at the distal end thereof, the needle 110 defining one end of a central lumen 190 that is operatively coupled on its other end with an aspiration pump (not shown) associated with surgical console 9. Thereby, the aspiration pump aspirates fluid from phaco tip 110a along aspiration pathway 124.

The proximal end of needle 110 is coupled with horn 250, which has its proximal end coupled with piezoelectric elements 202 (shown as piezoelectric discs). Horn 250, piezo-elements 202, and proximal portion of the needle 110 are enclosed within handpiece casing 270, which additionally provides irrigation pathway 226. Irrigation pathway 226 is coupled with an irrigation source (not shown) associated with console 9.

Horn 250 is typically an integrated metal, such as titanium, structure and may include an O ring 260 just before horn 250 tapers to fit with needle 110 at the horn's distal end. The O ring 260 snugly fits between horn 250 and casing 270. The O ring 260 may seal the proximal portion of horn 250 from the irrigation pathway 226. By way of example, descriptions of handpieces known in the art are provided in U.S. Pat. No. 6,852,092 to Kadziauskas et al. and U.S. Pat. No. 5,843,109 to Mehta et al., which are hereby incorporated by reference in their entirety.

In preparation for operation, sleeve 112 is typically added to the distal end of the handpiece 100, covering the proximal portion of the needle 110 (and thus exposing the distal tip 110a of the needle) and the distal end of the irrigation pathway 226, thereby defining an irrigation port 226a just before the distal tip 110a of needle 110. Needle 110 and a portion of the sleeve 112 may then be inserted through the cornea of the eye to reach the cataract lens.

During operation, the irrigation pathway 226, the eye's chamber and the aspiration pathway 124 form a fluidic circuit, wherein irrigation fluid enters the eye's chamber via the irrigation port 226a, and is then aspirated through the aspiration pathway 124 via port 124a along with other materials that the surgeon desires to aspirate out, such as the cataract lens. If, however, the materials, such as the cataract lens, are too hard and/or large to be aspirated through the aspiration pathway 124, then the distal end of the needle 110 is ultrasonically vibrated and applied to the lens material in order to emulsify the lens material into a size and state that can be successfully aspirated.

Needle 110 is ultrasonically vibrated by applying electric power to the piezoelectric elements 202, which, in turn, cause the horn 250 to ultrasonically vibrate, which, in turn, ultrasonically vibrates needle 110. The electric power is defined by a number of parameters, such as signal frequency and amplitude, and if the power is applied in pulses, then the parameters can further include pulse width, shape, size, duty cycle, amplitude, and so on. These parameters are controlled by a control unit (not shown), and example control of these parameters is described in U.S. Pat. No. 7,169,123 to Kadziauskas et al., which is hereby incorporated by reference in its entirety

In a traditional phacoemulsification system 1, the applied electric power has a signal frequency that causes the element(s) 202, horn 250, and needle 110 assembly to vibrate at a mechanically resonant frequency. This causes the needle 110 to vibrate in at least the longitudinal direction, i.e., along the z axis as shown, with a maximum range of motion, which may be considered to be the state in which the needle's cutting efficacy is at its maximum.

Non-longitudinal operating modes may also include torsional and transversal modes. Torsional phacoemulsification designs involve operating the needle tip in a rotational manner. The torsional mode produces a to and fro motion about the longitudinal axis of needle 110. Transversal ultrasound phacoemulsification technology enables operation of the needle tip with side-to-side transversal movements, which may be combined with traditional forward-and-back longitudinal stroke action. The tip motion realized from combining these two operating modes produces a cutting tip motion that follows an elliptical pattern at the phaco tip 110a.

Thus, existing phacoemulsification systems typically enable the phaco tip 110a to ultrasonically vibrate at least in a direction of the longitudinal axis, i.e., axis Z as shown, of the handpiece 100, and may additionally enable the phaco tip 110a to ultrasonically vibrate in a direction that is transverse of the longitudinal axis, i.e., along the x and/or y axes. Existing systems may also enable alternating between these directional vibrations by alternating between two different pulses of energy applied to the handpiece 100.

In yet further examples, the distal and proximal portions of handpiece 100 may be coupled with each other, such as at either end of a central body. This central body may provide the coupling by inclusion of a coupler, which may be or include a swivel feature that allows the distal and proximal portions of handpiece 100 to rotate independently from each other about axis z.

The handpiece 100, at the proximal end thereof, may have inputs and/or outputs, such as for the aspiration and irrigation lines discussed above, associated with performance of the surgery. These inputs/outputs may be provided to/from surgical console 9. That is, these inputs and outputs may be fluidically and/or electrically communicative with surgical console 9, and console 9 is provided to monitor and/or control the surgery performed using handpiece 100. Accordingly, the surgical console 9 may include one or more user interfaces, such as a graphical user interface (GUI) 11, such as may provide information related to the surgery to a user, and such as may receive control inputs 11a from the user.

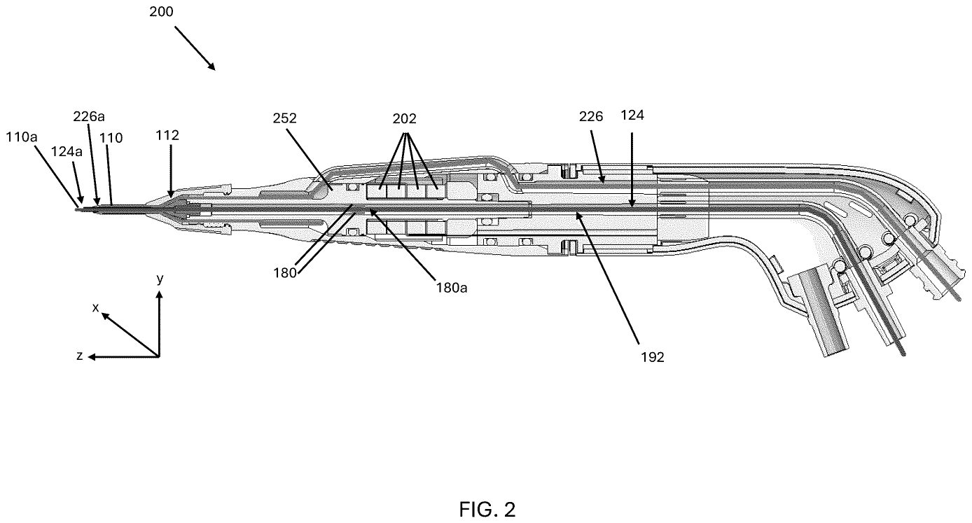

FIG. 2 is a cross-sectional illustration of an exemplary surgical handpiece 200. Again, illustrated is needle 110, having phaco tip 110a at the distal end of handpiece 200 for performing surgery, such as phacoemulsification. It is noted that the surgical handpiece 200 includes one or more of the same or similar features as the surgical handpiece 100. As such, the like reference numerals of the surgical handpiece 200 and the surgical handpiece 100 represent like parts and assemblies, and as such, a description of these parts and assemblies is not repeated.

Also shown are piezoelectric elements 202 for driving needle 110. Although four piezoelectric elements 202 are shown in cross-section circumferentially or substantially circumferentially about the interior of the handpiece 200, it will be appreciated that any number or type of piezoelectric elements 202 may provide ultrasonic energy to phaco tip 110a.

Central lumen 192 of handpiece 200 may typically be a single body, or may be a limited number of bodies, so as to have limited points of connectivity. This allows for the maintenance of substantial smoothness, straightness, and uniformity along its length. This is typically the case in order to prevent discontinuities in central lumen 192, which historically were believed to cause clogging in the aspiration pathway 124. Thus, it is of particular note that central lumen 192 of handpiece 200 includes a constriction zone 180a having intentionally-defined, structural non-uniformities/constrictions 180, for example in the form of a convex ramp extending circumferentially from the inside diameter of central lumen 192 towards the axis running longitudinally through the center of central lumen 192. During the phacoemulsification procedure, an aspiration pump is activated to pull fluid and particles from the eye through aspiration port 124a and aspiration pathway 124.

Similarly, during the phacoemulsification procedure, irrigation fluid is provided to the eye via irrigation pathway 226, sleeve 112, and irrigation port 226a from an irrigation fluid source (not shown) and sleeve 112.

It is of note that all portions of the handpiece 200 within ultrasonic horn 252, i.e., all portions of the handpiece 200 subject to the vibrational effects of piezoelectric element(s) 202, experience repetitive movement along, above and below, and/or around axis z, as discussed throughout. For example, upon the oscillation movement responsive to piezoelectric element(s) 202, central lumen 192 of aspiration pathway 124 may stretch (as longitudinal movement occurs toward the distal end of phaco tip 110a) and release (as movement occurs away from the distal end of phaco tip 110a). Accordingly, and based in part on the nature of the construction and the diameter of the lumen 192, the constriction zone 180a may expand and contract with the oscillation movements.

The translation of the ultrasonic energy to needle movement for emulsification may particularly occur at a “resonance mode” of piezoelectric elements 202. Particularly when resonance occurs, central lumen 192 of aspiration pathway 124 is subjected to ultrasonic energy and may thus stretch and thin out on one movement, and then shrink and expand circumferentially on the next movement, as referenced above. At the nodal point of the resonance mode, there is thus significant ongoing radial expansion and contraction of at least central lumen 192 of fluidic pathway 124.

As discussed, oscillation may be principally carried in the “forward” direction, i.e., from piezoelectric elements 202 towards needle 110 from ultrasonic horn 252, at least in part by the structure(s) of and around central lumen 192. In particular, the oscillation from the piezoelectric elements 202 may occur in ultrasonic horn 252, in which secondary emulsification occurs. The horn 252 translates the vibration of the piezoelectric elements 202 to the needle 110, in which the longitudinal and/or transversal oscillation may be seen at the needle tip 110a. Central lumen 192 runs centrally through handpiece 200, from aspiration port(s) 124a through the body of handpiece 200 to provide a fluid pathway to fluidically connect from the needle tip 110a through handpiece 200 to an aspiration collection receptacle outside of handpiece 200 (not shown). Of note, central lumen 192 may also be included in ultrasonic horn 252 solely in the portion thereof between elements 202 and needle 110. Thus, central lumen 192 may extend the entire length, substantially the longitudinal length, or only part of the length, of handpiece 200. At least the inner surface of the central lumen 192 expands and contracts with the longitudinal and/or transversal oscillation.

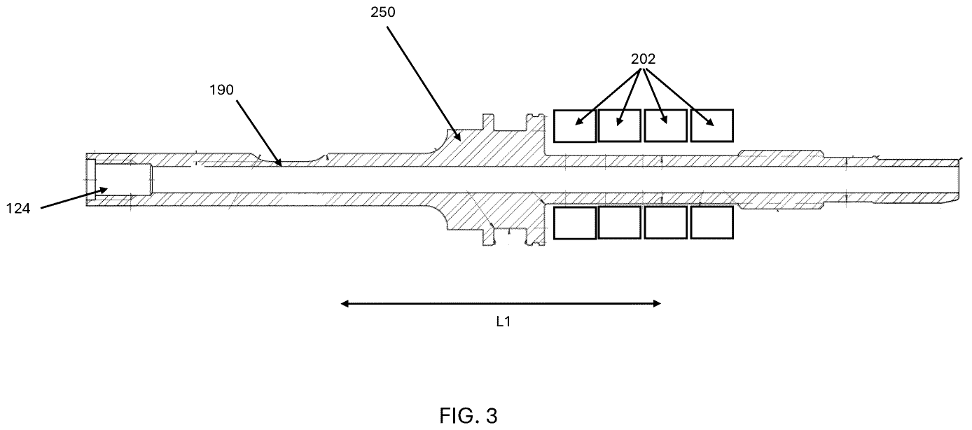

FIG. 3 is a cross-sectional illustration of a central portion of a typical ultrasonic horn 250. More particularly, shown is central lumen 190 arranged to accommodate aspiration pathway 124 as it passes through handpiece 100, as discussed throughout. More specifically, central lumen 190 of FIG. 3 passes at least through piezoelectric element(s) 202.

The central lumen 190 in FIG. 3 is a typical, known lumen that is straight, smooth, and uniform along its length in an effort to avoid imparting any conformational effect to the aspiration pathway 124 running therethrough. This traditional design is based, in part, on the nature of aspiration pathway 124 and the consequent tendency of pathway 124 to clog at any imperfections, bends, curves, or other structural inconsistencies that might be present in central lumen 190. It thus was historically believed that such structural inconsistency within central lumen 190 causes constriction points within aspiration pathway 124, which would in turn cause the undesirable effect of clogging by particles and fluid being aspirated from needle 110 through aspiration pathway 124. Accordingly, it is generally accepted that the illustrated design of lumen 190, which is straight, smooth and uniform along its length, minimizes the formation of clogs within aspiration pathway 124 running therethrough.

It will thus be understood that, in the traditional design of FIG. 3, the vibrational energy supplied by the ultrasonics is targeted to emulsify only at the phaco tip (not shown in FIG. 3). This is notwithstanding the fact that the ultrasonic energy is provided throughout the axial length L1 of the horn 250, including along at least the needle-side length of the central lumen 190. However, the smooth, featureless nature and straightness of central lumen 190 of FIG. 3 provides nothing against which the ultrasonic energy may force particles to further emulsify those particles within the central lumen 190 beyond the emulsification provided at the phaco tip. In contrast to the known art, in the examples contemplated herein, vibrational energy from ultrasonic elements positioned along the handpiece 200 is also used in constriction zones 180a along the axial length L2 of the central lumen 192 to provide additional points of emulsification of aspirated particles.

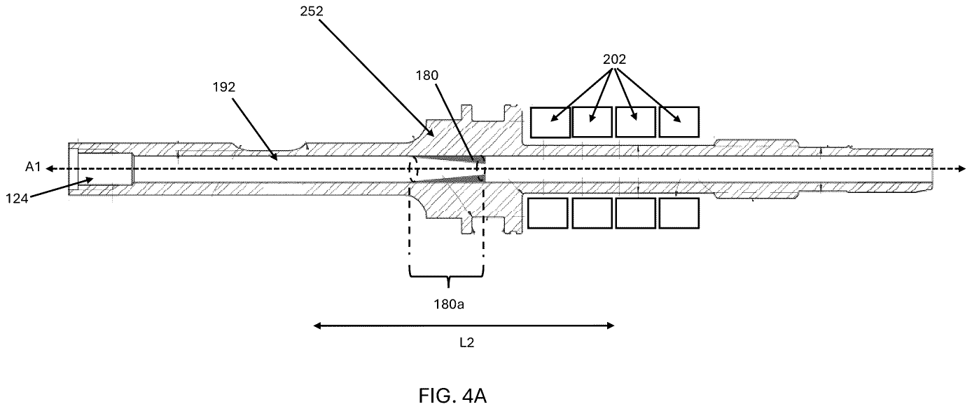

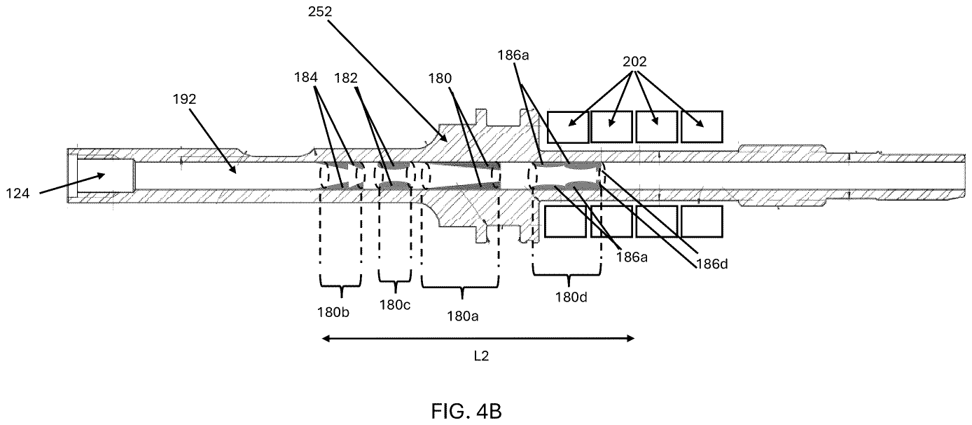

FIG. 4A illustrates an example of central lumen 192 distinct from the lumen 190 shown in FIG. 3, and akin to the non-limiting example of FIG. 2. More particularly, central lumen 192 includes one or more intentionally-designed, structural non-uniformities 180 extending from the inner diameter surface of central lumen 192. Aspiration pathway 124 through the central lumen 192 of FIG. 4A will thus experience conformance of the cross-section thereof to the designed non-uniformity 180 during ultrasonic performance, as discussed throughout. It should be appreciated that the intentional inclusion of a structural non-uniformity 180 within central lumen 192 is highly unconventional and counterintuitive to what is traditionally understood in the pertinent art, as discussed throughout.

More particularly, the areas/points/zones of constriction 180a formed along aspiration pathway 124 by constriction(s) 180 provide additional emulsification areas within aspiration pathway 124. That is, as pathway 124 experiences at least the longitudinal movement imparted by piezoelectric elements 202 as discussed throughout, constriction zone 180a expands and contracts. This periodic expansion and contraction may cause a modified fluidics flow, and may also bring a force down upon any particles present in the cross section of the constriction zone 180a, due at least to aspiration pathway 124 periodically constricting at constriction zone(s) 180a during vibration of lumen 192. As will be appreciated, this high speed application of vibrational forces serves to repeatedly expand and contract pathway 124, which breaks up any particles within the constriction zone 180a. Further, the modified flow at the constriction zone 180a may also increase the exposure time for the particles undergoing this secondary “blast” of emulsification energy by piezoelectric elements 202 by holding those particles in place.

Within the central lumen 192, the amplitude and makeup of the afore-discussed longitudinal, transverse, and/or radial vibration modes varies along the length of the lumen 192, and with the amplitude and makeup of operation of the piezoelectric elements 202, as detailed above. The piezoelectric elements 202 provide at least a longitudinal and a transversal oscillation of the needle 110 along a center axis A1 of the lumen 192. The horn 252 translates the vibration of the piezoelectric elements 202 to the needle 110, in which the longitudinal and a transversal oscillation are seen at the needle tip 110a. At least the inner surface of the lumen 192 expands and contracts with the longitudinal and the transversal oscillation, thereby causing the constriction(s) 180 to oscillate as well. As such, the constriction zone or zones 180a may be designed and positioned so as to optimize the level and type of emulsifying energy that is imparted to the aspirated particles passing through central lumen 192 during operation of the piezoelectric elements 202.

In conjunction with the amplitude and makeup of the modes used for the secondary emulsification, further design considerations for the constriction zone or zones 180a include the number of constriction zone(s) and the geometry of constriction zone(s) 180a correspondent to constriction(s) 180, as well as the location of constriction zone(s) 180a within central lumen 192. One or multiple, such as two, three, four, or more constriction(s) 180, may be implemented, such as in individual stages of constriction(s) to provide one or more group-stage constriction zone(s) 180a, with each group having the same or distinct particular design features, to thereby increase the amount of secondary emulsification, and/or to act upon particles of decreasing size through each corresponding constriction zone 180a in a multi-step process. For example, as illustrated in FIG. 4B, central lumen 192 may include constriction zone 180a, constriction zone 180b, constriction zone 180c, and constriction zone 180d. It is noted that any number of constriction zones may be included in lumen 192. In some cases, the constriction zones may include a same geometry or shape. In other cases, the constriction zones may include a combination of geometries and shapes, as illustrated in FIG. 4B.

To achieve the desired constriction zone(s) 180a for a given design, the geometry of the constrictions 180 may be tapered, such as on one or both ends, or may be concave, convex, stepped, or any number of other geometries, and the same or a different shape, shapes, or symmetry may present on one or both ends of the constriction 180. As such, the constriction may be ramped on one or both sides thereof, and may have a vertical face or faces, variable slopes, and so on, by way of non-limiting example. The constriction(s) 180 may be circumferentially continuous, discontinuous, partial, or periodic around the inner diameter of central lumen 192. Yet further, the constriction zones 180a may comprise periodic structural variations/designed inconsistencies in the inner diameter of central lumen 192, and thus along the inner diameter of aspiration pathway 124 within the area of the lumen 192 affected by piezoelectric elements 202; or, to the extent separate tubing is encompassed within the inner diameter of lumen 192, constrictions 180 may be provided on the inner diameter of the separate tubing, on the inner diameter of lumen 192, or on combinations thereof.

By way of particular example, a constriction, such as constriction 182 of constriction zone 180c, may be a ramped up-and-down convexity that is uniform around the entire circumference of the inner surface of the central lumen 192, as illustrated in FIG. 4B. Alternatively, the constriction, such as constriction 184 of constriction zone 180b, may be a “saw tooth” having a flat, thin, vertical-faced front, and a ramped back, as illustrated in FIG. 4B. In the latter example, a plurality of such “saw teeth” may be placed periodically (rather than continuously) around the circumference of the inner diameter of the central lumen 192. Alternatively, “fins” may be provided periodically, having ramped front portions and rear portions with thin presenting and trailing cross-sections, but such as with a larger cross section presented at the center thereof. Multiple rows of fins and/or saw teeth may be presented to aspirated particles moving longitudinally down the central lumen 192 away from the needle 110. Of note, saw teeth and/or fins may be formed such that they lean or vibrate as ultrasonic energy is imparted.

Additionally, rather than presenting a small initial cross-section to particles moving down central lumen 192, the constriction 180 may instead present a relatively flat and broad vertical and/or horizontal face, such as with a ramped rear portion. Yet further, the constriction 180 may present a series of bumps, such as constrictions 186a of constriction zone 180d, having a small presenting cross-section that ramps progressively to a blunt back section, such as constriction 186a of constriction zone 180d, having a broad horizontal and vertical cross section, such as to provide greater particle crushing capability at the rear of the constriction zone, such as constriction zone 180d, upon contraction of aspiration pathway 124, as illustrated in FIG. 4B.

In general, with regard to the geometry of constriction(s) 180, the greater the surface area, the more surface is present to apply crushing and ultrasonic vibrational forces in constriction zone 180a so as to further emulsify particles. However, increases in surface area must be balanced with decreased cross-sectional area of the fluidics path, which may lead to an increase in the likelihood of clog development. Accordingly, the design of the structural constrictions 180 which cause constriction zone(s) 180a is made to increase the emulsifying surface area in a manner that ultimately results in a net-reduction of clogging, contrary to the beliefs of the known art, rather than an increase in clogging.

As mentioned above, surface area and cross-section are geometric considerations which also may correspond to the ultrasonic mode under which the constriction(s) 180 is to perform its function. By way of non-limiting example and as will be further appreciated by the skilled artisan in light of this disclosure, a flat vertically extending face will work best in a longitudinal motion ultrasonic mode, creating areas of increased cavitation, while a circumferentially uniform convex bump, or a series of convex bumps, will have maximum effect by imparting blunt force to crush particles during a radial ultrasonic mode of operation.

The location of the constriction(s) 180 may thus be designed in conjunction with the constriction geometry to optimize secondary emulsification performance, either in a single secondary emulsification or in a multi-step secondary emulsification. For example, a stepped constriction facilitates energy transfer primarily from its stepped “face” in the longitudinal direction, and therefore may be located near a longitudinal anti-node to maximize force amplitude. In contrast and also by way of example, a tapered constriction may impart less longitudinal energy, but imparts greater energy from the transverse and radial vibration modes. As such, a tapered constriction may be positioned near a transverse anti-node and/or at a longitudinal node (which typically corresponds with a radial anti-node), by way of non-limiting example.

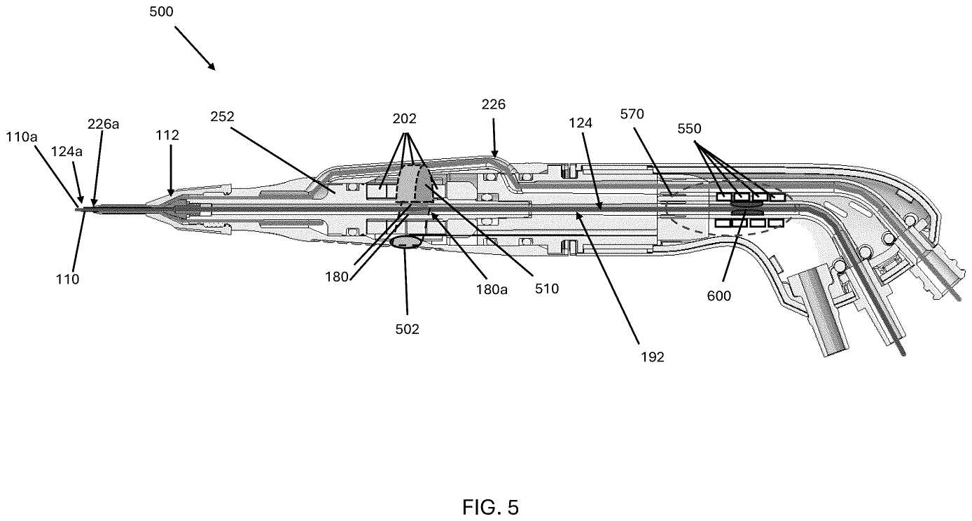

FIG. 5 is a cross-sectional illustration of a handpiece 500 similar to that shown in FIG. 2. It is noted that the surgical handpiece 500 includes one or more of the same or similar features as the surgical handpiece 200. As such, the like reference numerals of the surgical handpiece 500 and the surgical handpiece 200 represent like parts and assemblies, and as such, a description of these parts and assemblies is not repeated. The example of FIG. 5 includes several additional and alternative exemplary features.

More particularly, FIG. 5 illustrates an actuator, such as an actuating roller 502, which actuates the constriction zone(s) 180a. As shown, a finger actuating actuator 502 rotates a semi-circular constriction 180 forward around guide 510, thereby progressively inserting ramped convex constriction 180 partially around the inner diameter of central lumen 190. Likewise, actuator 502 may comprise a twistable handle, an inwardly rotatable threaded element, a push button, or the like, by way of non-limiting example. Accordingly, the central lumen 192 may include one or more regions that are configured to transition from a non-constricted state to a constricted state, depending on whether or not constriction 180 is actuated.

In an additional feature of the illustrated examples, a second ultrasonic transducer 550 may be provided, such as to supply additional or supplementary vibrational energy, vibrational modes, and so on. It will be appreciated that this second transducer 550 may be provided anywhere in the fluidics path along the aspiration pathway 124, such as in addition to, in conjunction with, or separately from the effects of horn 252. Within the zone/horn 570 affected by this second transducer 550 may also be provided additional constriction(s) 600. This additional zone/horn 570 may provide further emulsifying of particles passing through aspiration pathway 124. It will be understood that the second transducer 550 may require power for operation, and that this power may be provided by the same power supply as provides power to horn 252, or the power to second transducer 550 may be provided by an alternative power supply.

Accordingly, contemplated herein is a surgical handpiece that includes a plurality of emulsification zones, including a first emulsification zone positioned at the needle, and at least a second emulsification zone positioned within a constriction zone along the aspiration pathway through the handpiece. In some examples, the handpiece includes at least one ultrasonic transducer that directs ultrasonic energy to both the first emulsification zone and the second emulsification zone. In some examples, the handpiece includes a first ultrasonic transducer configured to direct ultrasonic energy to the first emulsification zone and a second ultrasonic transducer configured to direct ultrasonic energy to at least one additional second emulsification zone.

### EXAMPLES

### Example 1

A surgical handpiece (200) comprising: a body, wherein the body has a proximal end, a distal end, and a longitudinal axis, wherein the distal end is configured to be coupled with a needle (110); a horn (252), wherein the horn comprises a lumen (192) and is configured to couple with the needle; one or more piezoelectric elements (202), wherein the one or more piezoelectric elements are coupled with the horn and configured to ultrasonically vibrate a distal end of the needle to emulsify material; an aspiration channel extending from the distal end of the needle and through the lumen of the horn creating an aspiration pathway (124) along the longitudinal axis; and at least one constriction (180) on an inner surface of the lumen of the horn and into the aspiration pathway, wherein the at least one constriction is configured to emulsify the material proximally along the aspiration pathway from the distal end of the needle in response to the one or more piezoelectric elements.

### Example 2

A surgical handpiece (200), comprising: a horn (252) configured to receive ultrasonic energy from a first piezoelectric transducer (202) to drive a needle (110); a lumen (192) comprising an aspiration pathway (124), wherein the lumen is configured to receive effects of the first piezoelectric transducer and is configured to pass aspirated particles therethrough; and at least one constriction (180) on an inner surface of the lumen, wherein the at least one constriction is configured to expand into and contract away from the aspiration pathway in response to activation of the first piezoelectric transducer.

### Example 3

A system (1) for providing a plurality of emulsification zones (180a) for particles produced during a surgery, comprising: a surgical console (9) including a graphical user interface (GUI) (11) configured to receive control inputs (11a) for the surgery; a surgical handpiece (200) having a plurality of fluidic (124, 226) and electrical lines communicative with the surgical console, the surgical handpiece comprising: a body, wherein the body has a proximal end, a distal end, and a longitudinal axis, wherein the distal end is configured to be coupled with a needle (110); a horn (252), wherein the horn comprises a lumen (192) and is configured to couple with the needle; one or more piezoelectric elements (202) communicative with the electrical line and response to one of the control inputs, wherein the one or more piezoelectric elements are coupled with the horn and configured to ultrasonically vibrate a distal end of the needle to emulsify material; one of the fluidic lines communicative with an aspiration channel extending from the distal end of the needle and through the lumen of the horn creating an aspiration pathway (124) along the longitudinal axis, the aspiration pathway being fluidically configured to aspirate the material responsive to another one of the control inputs; and at least one constriction (180) extending from an inner surface of the lumen of the horn and into the aspiration pathway, wherein the at least one constriction is configured to emulsify the material proximally along the aspiration pathway from the distal end of the needle in response to the one or more piezoelectric elements.

### Example 4

A surgical handpiece (200) for emulsifying material, comprising: a horn (252) configured to receive ultrasonic energy from a first transducer (202); a lumen (192) extending at least partially through the horn, the lumen having at least one fluid constriction zone (180a); and a needle (110) extending from the lumen, the needle comprising an aspiration port (124a) fluidly connected to the lumen; wherein the first transducer is configured to direct ultrasonic energy via the horn to a first emulsification zone at the needle and to a second emulsification zone in the at least one fluid constriction zone of the lumen.

### Example 5

The surgical handpiece of any one of examples 1-4, wherein the one or more piezoelectric elements (202) provides at least a longitudinal and a transversal oscillation of the needle (252) along a center axis of the lumen (192), and wherein at least the inner surface of the lumen expands and contracts with the longitudinal and the transversal oscillation.

### Example 6

The surgical handpiece of any one of examples 1-5, wherein the horn (252) is configured to receive ultrasonic energy from the one or more piezoelectric elements (202).

### Example 7

The surgical handpiece of any one of examples 1-6, wherein one or more piezoelectric elements (202) comprise a disc shape and are configured to surround an outer surface of the horn.

### Example 8

The surgical handpiece according to any of examples 1-7, wherein the at least one constriction (180) extends fully, partially, or periodically from around the inner surface of the lumen (192).

### Example 9

The surgical handpiece according to any of examples 1-8, wherein the at least one constriction (180) is at least partially convex, concave or saw tooth in shape.

### Example 10

The surgical handpiece according to any of examples 1-9, wherein the at least one constriction (180) includes multiple stages (180a) of constrictions progressing proximally along the inner surface of the lumen (192).

### Example 11

The surgical handpiece according to any of examples 1-10, wherein the surgery is phacoemulsification.

### Example 12

The surgical handpiece according to any one of examples 1-11, further including a second transducer (550) configured to provide a third emulsification zone in at least a second fluid constriction zone (570) of the lumen (192).

### Example 13

The surgical handpiece according to example 12, further including a second horn associated with the second transducer (550) and being operable upon the second fluid constriction zone.

Although the examples described herein mainly address phacoemulsification procedures, the methods and systems described herein can also be used in other medical applications. Further, the various examples described above are provided by way of illustration only, and should not be construed to limit the claims attached hereto, including to any specific type of surgical procedure.

Further, the descriptions of the disclosure are provided to enable any person skilled in the art to make or use the disclosed examples. Various modifications to the disclosure will be readily apparent to those skilled in the art based on this disclosure, and the generic principles defined herein may be applied to other variations without departing from the spirit or scope of the disclosure. Thus, the disclosure is not intended to be limited to the examples and designs described herein, but rather is to be accorded the widest scope consistent with the principles and novel features claimed as follows.

What is claimed is:

1. A surgical handpiece, comprising:
a body, wherein the body has a proximal end, a distal end, and a longitudinal axis, wherein the distal end is configured to be coupled with a needle;
a horn, wherein the horn comprises a lumen and is configured to couple with the needle;
one or more piezoelectric elements, wherein the one or more piezoelectric elements are coupled with the horn and configured to ultrasonically vibrate a distal end of the needle to emulsify material;
an aspiration channel extending from the distal end of the needle and through the lumen of the horn creating an aspiration pathway along the longitudinal axis; and
at least one constriction extending from an inner surface of the lumen of the horn and into the aspiration pathway, wherein the at least one constriction is configured to emulsify the material proximally along the aspiration pathway from the distal end of the needle in response to the one or more piezoelectric elements.

2. The surgical handpiece of claim 1, wherein the one or more piezoelectric elements provides at least a longitudinal and a transversal oscillation of the needle along a center axis of the lumen, and wherein at least the inner surface of the lumen expands and contracts with the longitudinal and the transversal oscillation.

3. The surgical handpiece of claim 1, wherein the horn is configured to receive ultrasonic energy from the one or more piezoelectric elements.

4. The surgical handpiece of claim 3, wherein the one or more piezoelectric elements comprise a disc shape and are configured to surround an outer surface of the horn.

5. The surgical handpiece of claim 1, wherein the at least one constriction extends fully circumferentially from around the inner surface of the lumen.

6. The surgical handpiece of claim 1, wherein the at least one constriction extends partially circumferentially from around the inner surface of the lumen.

7. The surgical handpiece of claim 1, wherein the at least one constriction extends periodically from around the inner surface the lumen.

8. The surgical handpiece of claim 1, wherein the at least one constriction is one of at least partially convex or partially concave in shape.

9. The surgical handpiece of claim 1, wherein the at least one constriction comprises a saw tooth shape.

10. The surgical handpiece of claim 1, wherein the at least one constriction comprises multiple stages of constrictions progressing proximally along the inner surface of the lumen.

11. A surgical handpiece, comprising:
a horn configured to receive ultrasonic energy from a first piezoelectric transducer to drive a needle;
a lumen comprising an aspiration pathway, wherein the lumen is configured to receive effects of the first piezoelectric transducer and is configured to pass the aspirated particles therethrough; and
at least one constriction on an inner surface of the lumen, wherein the at least one constriction is configured to expand into and contract away from the aspiration pathway in response to activation of the first piezoelectric transducer.

12. The surgical handpiece of claim 11, wherein the at least one constriction is one of at least partially convex or partially concave in shape.

13. The surgical handpiece of claim 11, wherein the first piezoelectric transducer provides at least a longitudinal and a transversal oscillation of the needle, and wherein the expanding and contracting of the lumen occurs with the longitudinal and transversal oscillation.

14. A system for providing a plurality of emulsification zones for particles produced during a surgery, comprising:
a surgical console including a graphical user interface (GUI) configured to receive control inputs for the surgery;
a surgical handpiece having a plurality of fluidic and electrical lines communicative with the surgical console, the surgical handpiece comprising:
a body, wherein the body has a proximal end, a distal end, and a longitudinal axis, wherein the distal end is configured to be coupled with a needle;
a horn, wherein the horn comprises a lumen and is configured to couple with the needle;
one or more piezoelectric elements communicative with the electrical line and responsive to one of the control inputs, wherein the one or more piezoelectric elements are coupled with the horn and configured to ultrasonically vibrate a distal end of the needle to emulsify material;
one of the fluidic lines communicative with an aspiration channel extending from the distal end of the needle and through the lumen of the horn creating an aspiration pathway along the longitudinal axis, the aspiration pathway being fluidically configured to aspirate the material responsive to another one of the control inputs; and
at least one constriction extending from an inner surface of the lumen of the horn and into the aspiration pathway, wherein the at least one constriction is configured to emulsify the material proximally along the aspiration pathway from the distal end of the needle in response to the one or more piezoelectric elements.

15. The system of claim 14, wherein the surgery comprises phacoemulsification.

16. The system of claim 14, wherein the one or more piezoelectric elements comprise a disc shape and are configured to surround an outer surface of the ultrasonic horn.

17. The system of claim 14, wherein the at least one constriction comprises multiple stages of constrictions progressing proximally along the inner surface of the lumen.

18. A surgical handpiece for emulsifying material, comprising:
a horn configured to receive ultrasonic energy from a first transducer;
a lumen extending at least partially through the horn, the lumen having at least one fluid constriction zone; and
a needle extending from the lumen, the needle comprising an aspiration port fluidly connected to the lumen;
wherein the first transducer is configured to direct ultrasonic energy via the horn to a first emulsification zone at the needle and to a second emulsification zone in the at least one fluid constriction zone of the lumen.

19. The handpiece of claim 18, further comprising a second transducer configured to provide a third emulsification zone in at least a second fluid constriction zone of the lumen.

20. The handpiece of claim 19, further comprising a second horn associated with the second transducer and being operable upon the second fluid constriction zone.
